(19) United States
(12) Patent Application Publication
(10) Pub. No.: US 2020/0101187 A1
(43) Pub. Date: Apr. 2, 2020

(54) INFECTION CONTROL ATTACHMENT FOR STETHOSCOPES

(71) Applicant: Boehringer Technologies, LP, Phoenixville, PA (US)

(72) Inventor: Samantha Ashley Simmons, West Chester, PA (US)

(21) Appl. No.: 16/582,375

(22) Filed: Sep. 25, 2019

Related U.S. Application Data
(60) Provisional application No. 62/738,223, filed on Sep. 28, 2018.

(51) Int. Cl. A61L 2/26; A61L 2/18; A61B 7/02

(52) U.S. Cl. CPC A61L 2/26; A61B 7/02; A61L 2/18

(57) ABSTRACT

Disclosed is an attachment device including a bottle holder for sanitizing a portion of a stethoscope. The attachment device includes a bottle of a sanitizing liquid, and the bottle holder. The bottle holder includes at least two elongated fingers and an attachment member. The elongated fingers are spaced apart from each other and define an interior space configured for releasable receipt of the bottle. The bottle holder is configured so that the bottle can be readily inserted and removed from the interior space The attachment member is configured for mounting the bottle holder on the stethoscope.

### CROSS-REFERENCE TO RELATED APPLICATIONS

This utility application claims the benefit under 35 U.S.C. § 119(e) of Provisional Application Ser. No. 62/738,223 filed on Sep. 28, 2018 entitled Infection Control Attachment for Stethoscopes. The entire disclosure of the provisional application is incorporated by reference herein.

### FIELD OF THE INVENTION

This invention relates generally to infection control and more particularly to devices for mounting on a stethoscope to enable the stethoscope to be sanitized when desired by means of a sanitizing liquid held in the device.

### SPECIFICATION

### BACKGROUND OF THE INVENTION

Infection control is an important issue in modern hospitals, nursing homes, assisted living facilities, etc. With the increase in drug resistant bacteria, a greater emphasis is being placed on infection prevention than ever before. As new sources of contamination are identified, it has become apparent that a stethoscope that is used by medical personnel, if not cleaned, before use on each person to be examined with the stethoscope has the potential to carry some infection agent to the patient being examined and to the user of the stethoscope. Therefore, providing a readily available device for cleaning or otherwise sanitizing the stethoscope is valuable for infection control. A need exists for a device.

The subject invention addresses that need by providing a device in the form of an attachment that is configured to be mounted on the tubing of the stethoscope, and which includes a bottle or other container containing a sanitizing liquid which can be used to clean or otherwise sanitize the stethoscope.

### BRIEF SUMMARY OF THE INVENTION

One aspect of this invention is an attachment device for sanitizing a portion of a stethoscope. The stethoscope includes a tubing portion. The attachment device comprises a bottle, a pair of elongated fingers, and an attachment member. The hollow body is configured for holding a sanitizing liquid therein. The fingers of the pair of elongated fingers are spaced apart from each other and define an interior space between them. The interior space is configured for releasable receipt of the hollow body of the bottle. The interior space has an access opening in communication with the interior space through which the hollow body of the bottle can be inserted into the interior space and through which the hollow body of the bottle can be removed from the interior space. The attachment member is secured to one of the fingers and is configured for engaging the portion of the tubing of the stethoscope for mounting the bottle holder on the tubing portion of the stethoscope.

In accordance with another preferred aspect of the attachment device of this invention the pair of fingers are configured to be flexed to spread apart at the access opening to enable the hollow body of the bottle to be inserted through the access opening into the interior space and to be removed from the interior space through the access opening.

In accordance with another preferred aspect of the attachment device of this invention at least one of the pair of fingers has a free end in the form of an outwardly angularly flared skirt.

In accordance with another preferred aspect of the attachment device of this invention the attachment member comprises a clip.

In accordance with another preferred aspect of the attachment device of this invention the clip is a generally C-shaped member.

In accordance with another preferred aspect of the attachment device of this invention the attachment device comprises a detent for releasable engagement with a portion of the bottle.

In accordance with another preferred aspect of the attachment device of this invention the bottle holder comprises an integral unit.

In accordance with another preferred aspect of the attachment device of this invention the bottle holder is molded of a plastic material.

Another aspect of this invention is bottle holder configured for holding a bottle of a sanitizing liquid. The bottle holder is configured for mounting on a portion of a tubing of a stethoscope. The bottle holder comprises a pair of elongated fingers and an attachment member. The fingers of the pair of elongated fingers are spaced apart from each other and define an interior space between them. The interior space is configured for releasable receipt of the hollow body of the bottle. The interior space has an access opening in communication with the interior space through which the hollow body of the bottle can be inserted into the interior space and through which the hollow body of the bottle can be removed from the interior space. The attachment member is secured to one of the fingers and is configured for engaging the portion of the tubing of the stethoscope for mounting the bottle holder on the tubing portion of the stethoscope.

In accordance with another preferred aspect of the bottle holder of this invention the pair of fingers are configured to be flexed to spread apart at the access opening to enable the hollow body of the bottle to be inserted through the access opening into the interior space and to be removed from the interior space through the access opening.

In accordance with another preferred aspect of the bottle holder of this invention at least one of the pair of fingers has a free end in the form of an outwardly angularly flared skirt.

In accordance with another preferred aspect of the bottle holder of this invention the at least one of the fingers whose free end is in the form of said outwardly angularly flared skirt includes a detent adjacent the skirt for releasable engagement with a portion of the bottle.

In accordance with another preferred aspect of the bottle holder of this invention the attachment member comprises a clip.

In accordance with another preferred aspect of the bottle holder of this invention the clip is a generally C-shaped member.

In accordance with another preferred aspect of the bottle holder of this invention the bottle comprises a spray bottle.

In accordance with another preferred aspect of the bottle holder of this invention the bottle holder comprises an integral unit.

In accordance with another preferred aspect of the bottle holder of this invention the bottle holder is molded of a plastic material.

### BRIEF DESCRIPTION OF SEVERAL VIEWS OF THE DRAWINGS

FIG. 1 is an isometric view of one exemplary embodiment of an attachment device constructed in accordance with this invention shown mounted on a portion of the tubing of a conventional stethoscope, with the attachment device comprising an a bottle holder configured for holding a spray bottle containing a disinfecting or otherwise sanitizing liquid for selective application to the stethoscope;

FIG. 2 is an enlarged isometric view of the bottle holder shown in FIG. 1;

FIG. 3 is an enlarged top plan view of the bottle holder shown in FIG. 1;

FIG. 4 is an enlarged bottom plan view of the bottle holder shown in FIG. 1;

FIG. 5 is an enlarged distal end view of the bottle holder shown in FIG. 1; and

FIG. 6 is an enlarged sectional view taken along line 6-6 of FIG. 2.

### DETAILED DESCRIPTION OF A PREFERRED EMBODIMENT OF THE INVENTION

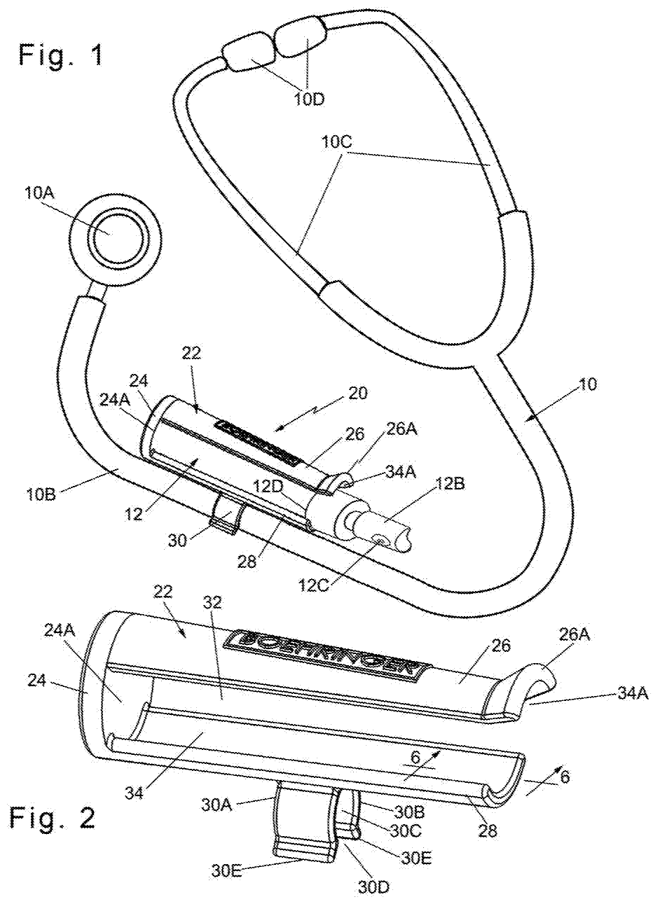

Referring now to the various figures of the drawing wherein like reference characters refer to like parts, there is shown in FIG. 1 an exemplary attachment device 20 configured for mounting (e.g., releasably securable mounting) on the tubing of a stethoscope 10. The stethoscope can be of any conventional construction. In the exemplary embodiment shown in FIG. 1 the stethoscope 10 basically comprises a chest piece 10A, e.g., a tunable diaphragm, mounted on the distal end of a tubing or tubing 10B, and a pair of ear-tubes or binaurals 10C, the proximal end of each of which terminating in a respective ear tip or ear plug 10D.

The attachment device 20 basically comprises a bottle holder 22 which is configured to be mounted on the tubing to releasably mount a bottle 12 containing a sanitizing agent on the stethoscope's tubing or tubing. The sanitizing agent is a liquid, which may constitute a disinfectant, a cleaning solution, or any other liquid useful for infection control. The bottle 12 can be of any type, although it is preferred to be a spray-type bottle. Thus, in the exemplary embodiment shown the bottle 12 has a hollow body 12A in which a disinfecting liquid (not shown) is disposed. The bottle includes a spray head 12B coupled to a pipe (not shown) extending into the sanitizing liquid. The spray head 12B is in the form of a depressible cap that is configured to be depressed by a user's finger when the bottle is in the hand of a user. That action causes a portion of the sanitizing liquid is pumped through the pipe to exit a nozzle 12C in the cap 12B to form a spray which the user can direct to the stethoscope chest piece 10A or any other portion of the stethoscope disinfect or otherwise clean it.

As will be described shortly the bottle holder 22 includes an interior space, e.g., a cavity or chamber, forming a socket configured for releasable receipt of the bottle 12, so that the bottle can be readily removed from the bottle holder and readily replaced therein. Moreover, the bottle holder 22 is compact in construction so that when mounted on the stethoscope with the bottle therein that combination does no interfere with normal use of the stethoscope. Thus, the user can readily remove the bottle 12 from socket of the bottle holder to clean/disinfect the stethoscope and then reinsert the bottle back into the socket, so that the bottle is readily available for subsequent use to clean/disinfect the stethoscope again when desired.

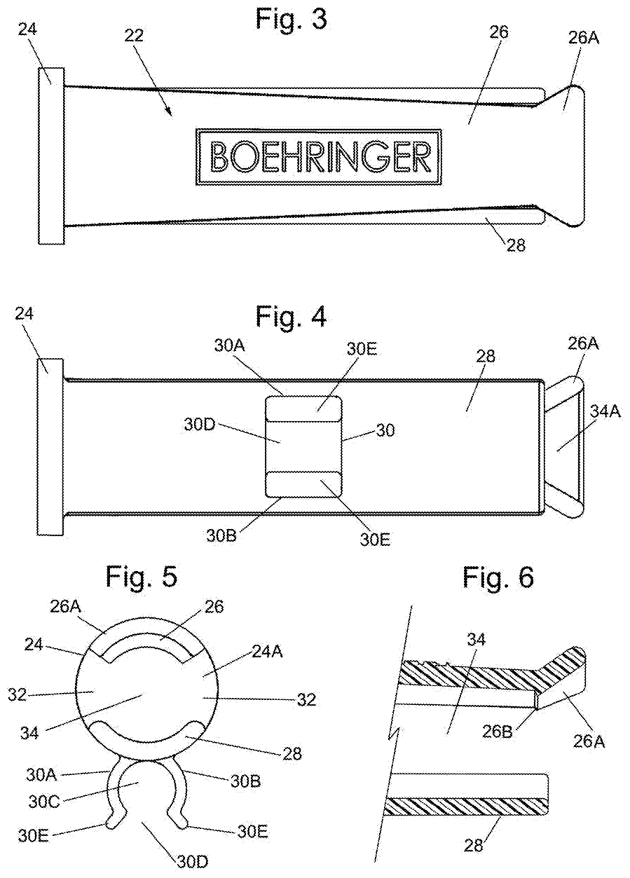

As best seen in FIGS. 2-6, the bottle holder 22 is an integral unit which basically comprises a base wall 24 and two elongate fingers 26 and 28 and a generally C-shaped clip 30. In accordance with a preferred embodiment of this invention the bottle holder is molded of any suitable plastic material. The base wall is of circular profile and includes an inner surface 24A. The finger 26 is an elongated member of arcuate cross section and which projects from the inner surface 24 of the base wall adjacent a first portion of the periphery of the base wall. The arcuate cross section of the finger 26 is less than 180 degrees. The finger 26 terminates at its distal end in an angularly outwardly flared skirt 26A. As best seen in FIG. 3, the finger 26 tapers downward slightly from the inner surface of the base wall 24 to the point at which the flared skirt 26A extends.

The finger 28 is also an elongated member of arcuate cross section of less than 180 degrees, and which projects from the inner surface 24 of the base wall adjacent a second portion of the periphery of the base wall. The second portion of the base wall is located diametrically opposed to the first portion of the base wall. Accordingly, as best seen in FIGS. 2 and 5 the fingers 26 and 26 are separated from each other by two longitudinally extending slots 32. The inner surfaces of those fingers define the interior space or socket 34 between them. Thus, the interior space or socket 34 is cylindrical and has an access opening 34A located adjacent the free ends of the fingers 26 and 28.

As best seen in FIG. 4, the distal end of the finger 28 does not include an angularly flared skirt. Moreover, it terminates at the point that the skirt 26A flares outward from the finger 26. It should be pointed out at this juncture that such a construction, while preferred, is not mandatory. Thus, the finger 28 could be constructed like the finger 26.

In any case the fingers 26 and 28 are configured so that they can be flexed or spread apart starting at their free distal ends, i.e., at the access opening, so that the bottle 12 can be inserted into the interior space or socket 34 via the access opening 34A. The flared skirt 26A facilitates the introduction of the bottle into the socket between the fingers since it serves to direct introduction or insertion of the bottle into the access opening 34A. The bottle 12 is selected to have a body having an outside diameter equal to or slightly larger than the inner diameter of the interior space or socket 34.

The bottle 12 is introduced into the interior space 34 as follows. The bottom end 12D of the bottle's body is juxtaposed so that it axially aligned with the access opening 34 to the socket 34. The bottle is then pushed towards the bottle holder along the central longitudinal axis of the socket. That action causes the bottom 12D of the bottle to engage the inner surface of the flared skirt 26A, which will guide the bottle into the socket. Further movement of the bottle in that direction causes the fingers 26 and 28 to flex outward to enable the body of the bottle to enter the distal end of the socket and slide down that space until the bottom of the bottle reaches the inner surface of the base wall or a point adjacent thereto. The natural spring bias of the fingers 26 and 28 will cause them to flex backward to frictionally engage the outer surface of the body of the bottle to hold the bottle in place like shown in FIG. 1. Moreover, as best seen in FIG. 1 a detent 26B extends outward from the undersurface of the skirt 26A at the point from which the skirt merges with the finger 26. That detent serves to snap-fit and engage a lip or ridge 12D (FIG. 1) on the bottle head to releasably hold the bottle within the socket 34. The bottle, however, can be readily removed from the bottle holder when desired by merely pulling on the portion of the bottle extending out of the access opening, e.g., the bottle's spray head 12, in the direction away from the bottle holder, whereupon the fingers 26 and 26 will flex apart to disengage the detent and thereby release the bottle.

As mentioned earlier the bottle holder 22 includes a generally C-shaped clip 30. That clip serves as the means for mounting the bottle holder onto any portion of the tubing 10B of the stethoscope. The clip 30 is best seen in FIGS. 2, 4 and 5. The clip 30 extends downward from the underside of the finger 28 at approximately the midpoint of the length of the bottle holder. The C-shaped clip is formed of an opposed pair of arcuate jaws sections 30A and 30B, which between them define a cylindrical interior space 30C having an entryway 30D. The free end of each of the arcuate jaw sections 30A and 30B is in the form of an outwardly extending tab 30E. The interior space 30C is equal to or slightly larger in diameter than the outer diameter of the stethoscope tubing 10B. The entryway to the interior space 30C is defined between the tabs 30E.

The mounting of the bottle holder 22 on the stethoscope tubing 10B is accomplished by juxtaposing the clip 30 along a portion of the stethoscope tubing onto which the bottle holder is to be mounted. Once that has been accomplished the user merely pushes on the bottle holder to cause the tabs 30E on the free ends of the jaws 30A and 30B to engage the tubing 10B. That action will cause the jaws to flex or spread apart to enable the tubing to pass through the entryway 30D into the interior space 30C, whereupon the jaws will flex back to their normal position, thereby securing the bottle holder onto the tubing.

As mentioned earlier the bottle holder 22 is compact, as is the bottle 12, so that the combination is not bulky which could interfere with normal use of the stethoscope when the attachment device is mounted thereto. For example, one particularly effective size of the bottle holder is one that has a length of approximately 2.73 inches, an outside diameter of 0.87 inch, with the interior space 34 having an inside diameter of 0.7 inch.

It should be pointed out that these dimensions are merely exemplary so that the bottle holder can be of another size (providing of course that the size of the central cavity is appropriate for the size of the body of the bottle). Moreover, the exemplary embodiment shown and described heretofore is merely one example of many attachment devices that can be constructed in accordance with this invention for releasably holding a bottle containing a sanitizing liquid. For example, the bottle holder can include more than two elongated fingers. Furthermore, while the exemplary embodiment of the attachment device is shown as including a clip to mount the bottle holder on a portion of the stethoscope tubing, other means can be used to mount the bottle holder thereon. Thus, a strip of adhesive tape can be used in place of the clip. So too, cooperating hook and loop fastening components, like VELCRO® fasteners, can be used in place of the clip. In fact, any means for mounting the bottle holder on the stethoscope tube can be used. That means can be either permanent or releasable, although it is preferred that it be releasable so that the bottle holder can be removed from the stethoscope to clean or otherwise sanitize the bottle holder if desired or necessary. Further still, the bottle need not be a spray bottle. Rather, it could be any type of bottle having a hollow interior holding a disinfecting or cleaning liquid, and a removable cap or cover to provide access to the disinfecting liquid so that a portion of the disinfecting liquid can be removed and applied to any portion of the stethoscope when desired. Further still,

Without further elaboration the foregoing will so fully illustrate my invention that others may, by applying current or future knowledge, adopt the same for use under various conditions of service.

I claim:

1. An attachment device for sanitizing a portion of a stethoscope, the stethoscope including a tubing portion, said attachment device comprising:
a bottle having a hollow body configured for holding a sanitizing liquid therein;
a bottle holder comprising:
at least two elongated fingers spaced apart from each other and defining an interior space between them, said interior space being configured for releasable receipt of said hollow body of said bottle, said interior space having an access opening in communication with said interior space through which said hollow body of said bottle can be inserted into said interior space and through which said hollow body of said bottle can be removed from said interior space; and
an attachment member secured to one of said fingers and being configured for engaging the portion of the tubing of the stethoscope for mounting said bottle holder on the tubing portion of the stethoscope.

2. The attachment device of claim 1, wherein said fingers are configured to be flexed to spread apart at said access opening to enable said hollow body of said bottle to be inserted through said access opening into said interior space and to be removed from said interior space through said access opening.

3. The attachment device of claim 2, wherein said at least one of said fingers has a free end in the form of an outwardly angularly flared skirt.

4. The attachment device of claim 1, wherein said attachment member comprises a clip.

5. The attachment device of claim 4, wherein said clip is a generally C-shaped member.

6. The attachment device of claim 2, wherein said attachment member comprises a clip.

7. The attachment device of claim 6, wherein said clip is a generally C-shaped member.

8. The attachment device of claim 1, wherein said attachment device comprises a detent for releasable engagement with a portion of the bottle.

9. The attachment device of claim 1, wherein said bottle holder comprises an integral unit.

10. The attachment device of claim 9, wherein said bottle holder is molded of a plastic material.

11. A bottle holder configured for holding a bottle of a sanitizing liquid, said bottle holder being configured for mounting on a portion of a tubing of a stethoscope, said bottle holder comprising:
at least two elongated fingers spaced apart from each other and defining an interior space between them, said interior space being configured for releasable receipt of said hollow body of said bottle, said interior space having an access opening in communication with said interior space through which said hollow body of said bottle can be inserted into said interior space and through which said hollow body of said bottle can be removed from said interior space; and
an attachment member secured to one of said fingers and being configured for engaging the portion of the tubing of the stethoscope for mounting said bottle holder on the tubing portion of the stethoscope.

12. The bottle holder of claim 11, wherein said fingers are configured to be flexed to spread apart at said access opening to enable said hollow body of said bottle to be inserted through said access opening into said interior space and to be removed from said interior space through said access opening.

13. The bottle holder of claim 12, wherein at least one of said fingers has a free end in the form of an outwardly angularly flared skirt.

14. The bottle holder of claim 11, wherein said attachment member comprises a clip.

15. The bottle holder of claim 14, wherein said clip is a generally C-shaped member.

16. The bottle holder of claim 12, wherein at least one of said fingers includes a detent for releasable engagement with a portion of said bottle.

17. The bottle holder of claim 13, wherein said at least one of said fingers whose free end is in the form of said outwardly angularly flared skirt includes a detent adjacent said skirt for releasable engagement with a portion of said bottle.

18. The bottle holder of claim 11, wherein said bottle comprises a spray bottle.

19. The bottle holder of claim 11, wherein said bottle holder comprises an integral unit.

20. The bottle holder of claim 19, wherein said bottle holder is molded of a plastic material.
